(19) United States
(12) Patent Application Publication
(10) Pub. No.: US 2013/0347144 A1
(43) Pub. Date: Dec. 26, 2013

(54) PROMOTERS AND METHODS FOR TRANSFORMING TUBERS AND TRANSFORMED TUBERS

(71) Applicant: Prairie Plant Systems Inc., Saskatoon (CA)

(72) Inventors: Ketan DOSHI, Saskatoon (CA); Natalia LOUKANINA, Saskatoon (CA)

(21) Appl. No.: 13/787,176

(22) Filed: Mar. 6, 2013

Related U.S. Application Data
(60) Provisional application No. 61/622,185, filed on Apr. 10, 2012.

(51) Int. Cl. C12N 15/82

(52) U.S. Cl. CPC C12N 15/8226
USPC 800/288; 536/24.1; 435/320.1; 435/419; 800/298; 435/69.1; 800/294

(57) ABSTRACT

The present disclosure relates to a plant promoter and a method of transforming Oxalis tuberosa. In detail the present disclosure relates to a plant promoter, a vector, including the promoter, a method of producing target protein using the vector, target protein produced by the method, a method for producing a transformed cell and a plant using the vector and a propagule of the plant.

### CROSS REFERENCE TO RELATED APPLICATIONS

This application claims the benefit of priority of U.S. Provisional Patent Application No. 61/622,185 filed Apr. 10, 2012, which is incorporated herein by reference in its entirety.

### FIELD

The present disclosure relates generally to methods of transforming plants and to plant promoters, as well as to transformed plants. More particularly, the present disclosure relates to plant promoters that direct of transcription to plant tubers and methods of producing transformed Oxalis tuberosa.

### BACKGROUND

It is a primary goal of research efforts in plant biotechnology to genetically engineer plants for the production of transgenic proteins in a process called molecular farming. It is also a goal to modify plants so that they have a new or improved trait or characteristic. In this regard, tubers (modified plant structures that are enlarged to store nutrients) are especially useful as vehicles for the production of transgenic proteins as they have the advantage of innate storing ability and stability. Examples of edible tubers that can be used for these purposes include but are not limited to potato, sweet potato, taro and Oca (Oxalis tuberosa).

The process of expressing a desired gene in a plant involves constructing a vector that comprises the gene of interest downstream of a promoter, introducing the vector into a plant, incorporating the gene stably into the genome of the plant, and expressing the gene in the plant. Generally the foreign DNA is integrated into nuclear DNA, however methods for the integration of foreign DNA into the plastid genome have been developed for some plant species (U.S. Pat. No. 5,451,513; and U.S. Pat. No. 5,693,507).

It is desirable to be able to direct gene expression to a specific organ, such as a tuber, in order to facilitate harvesting of proteins and to avoid protein production in other tissues which may have adverse effects on plant health or plant growth and which could raise regulatory concerns.

The identification of promoters and transformation methods specific to the type of plant to be transformed is critical in order to generate effective expression of a target gene.

Researchers have identified a number of promoters that are useful for the expression of genes in tubers. For example, potato tuber specific transcriptional regulation via the patatin potato storage protein promoter is described in U.S. Pat. No. 5,436,393 and U.S. Pat. No. 5,723,757.

An additional potato tuber specific promoter from the potato alpha amylase gene is described in U.S. Pat. No. 6,184,443. This promoter is induced by exposure to cold temperatures.

The sweet potato tuber-expressed sporamin gene promoter is described in U.S. Pat. No. 7,411,115 and the tarin storage protein promoter from Taro tubers by Guimaraes et al., 2001.

It is noted that sweet potato tubers are of root origin and the storage organ of Taro is botanically a corm or modified shoot structure, thus as such these promoters are not derived from stem tuber specific genes.

Many different procedures have been described that physically introduce foreign DNA into plant cells. A common strategy has been the “biolistic” acceleration of small dense carrier particles, such as particles of gold that are coated in foreign DNA, by what is known in the art as a “gene gun” (U.S. Pat. Nos. 4,945,050; 5,036,006; and 5,371,015). A variety of different “gene guns” for shooting DNA into plant cells have been developed (Ziolkowski, 2007; U.S. Pat. Nos. 5,976,880; 5,584,807). Other physically carriers such as tungsten “whiskers” or silicon carbide crystals have also been used to deliver foreign DNA by puncture of the cell wall creating channels for DNA entry (Asad, S et al. 2008; U.S. Pat. Nos. 5,302,523, 5,464,765; 7,259,016: 6,350,611).

Other physical approaches have included the micro-injection of DNA solutions directly into cells, (U.S. Pat. Nos. 4,743,548, 5,994,624) and production of pores in cellular membranes for DNA uptake with electric currents (U.S. Pat. No. 6,022,316). The removal of the external cell wall barrier and preparation of protoplasts facilitates the uptake of DNA directly from solution but in some instances regeneration of plants from protoplasts is challenging (U.S. Pat. Nos. 4,684,611; and 5,453,367).

By far the most widely practiced general method of achieving plant transformation has been by the use of disarmed strains of Agrobacteria (as reviewed in Gelvin, 2003). Agrobacterium tumefaciens and related soil bacteria naturally comprise a DNA plasmid (i.e. a T-DNA plasmid) that is physically mobilized into plant cells by bacteria proliferating in a wound site. The T-DNA plasmid has left and right border sequences that are required for integration of DNA into the plant host genome. Foreign DNA between the border sequences is thus selectively introduced into the host genome.

Naturally occurring Agrobacterium species introduce foreign DNA that comprises genes for the production of plant growth regulatory substances and uncommon amino acid metabolites known as opines. This results in the formation of a tumour at the site of infection that in addition to providing a refuge for the growth of Agrobacteria supplies specific nutrients beneficial to the bacteria. The formation of Crown Gall tumours, (or hairy root proliferation) by Agrobacterium sp. is an example of molecular parasitism. Naturally occurring plasmids have been modified, “disarmed” by removal of genes that cause tumour formation and support bacterial growth. The Ti plasmid was also modified to remove so-called virulence factors needed for DNA transfer. These factors were placed on a separate plasmid so that only selective recombinant DNA is added to the host plant cells and not the Vir genes. The technique of removal of the virulence factor DNA to a separate plasmid is known as “disarming” and resulted in the development of the preferred the binary transformation method (U.S. Pat. No. 4,940,838).

Initially, it was felt that Agrobacterium mediated transformation only occurred with dicot species however over time Agrobacterial strains that infect monocots were discovered and transformation using Agrobacterium was demonstrated (U.S. Pat. No. 5,591,616; and U.S. Pat. No. 7,060,876).

An important consideration for regeneration of transformed plants is the tissue targeted for biolistic or Agrobacterium mediated transformation. Tissue targets that have been shown to be useful for the regeneration of transformed plants include: leaf discs, stem segments, petioles, decapitated meristems, roots, flower buds and pollen. Any tissue can be used that can subsequently be regenerated into whole functional transgenic plants.

Previous attempts to produce transformed plants of Oxalis tuberosa or related Oxalis species have not been reported. Furthermore, although gene regulatory sequences have been described from many different genes with expression that varies from constitutive to cell or organ specific, few regulatory sequences from genes expressed in tubers have been described and none has been identified for Oxalis.

Thus there remains a need for promoters and methods for transforming Oxalis tuberosa. There is also a need for promoters and methods for transformation of tubers, specifically Oxalis tuberosa.

### SUMMARY

Generally, the present disclosure provides a method for transforming plants and a promoter that directs expression in tubers.

There is described herein a promoter comprising a nucleotide sequence which is SEQ ID NO:1 or a nucleotide sequence with 80% or greater identity to SEQ ID NO:1 which hybridizes to the complement of SEQ ID NO:1 under stringent conditions.

A vector comprising the promoter, a cell transformed with such a vector, a plant containing such a cell, a vegetative propagule of such a plant, and a method of producing a target protein in a tuber transformed with such a vector, are also described.

There is also described herein a method for producing transformed Oxalis tuberosa comprising: infecting Oxalis tuberosa nodal stem explant with agrobacterium expressing an expression vector to form an infected nodal explant; removing callus from infected nodal explant; inducing a bud on the callus; inducing shoot formation from the bud; and producing transformed Oxalis tuberosa from the shoot.

There is further described a method for producing transformed Oxalis tuberosa comprising: infecting Oxalis tuberosa stem nodal segments with agrobacterium expressing an expression vector; inducing a morphogenic callus; isolating a transformed morphogenetic stem callus; inducing a shoot bud from the morphogenetic stem callus by growing the bud in a medium comprising gibberellic acid; germinating the bud; inducing a shoot from the bud; germinating and elongating the shoot; rooting the shoot in rooting media; and producing a transformed plant.

Oxalis tuberosa plants produced according to the above methods are also described herein.

### BRIEF DESCRIPTION OF THE DRAWINGS

Embodiments will now be described, by way of example only, with reference to the Figures, wherein

FIG. 1A and FIG. 1B show the 5′ promoter sequence of the ocatin gene;

FIG. 2 is a schematic diagram of the constructs used for plant transformation;

FIG. 3 shows transient expression of GUS in potato and Oxalis tuberosa tubers;

FIG. 4 shows a lack of GUS expression in transgenic Oxalis tuberosa leaf and stem;

FIG. 5 shows stable expression of GUS in Oxalis tuberosa tubers;

FIG. 6 shows transient expression of GFP in Oxalis tuberosa shoots 4 weeks after transformation;

FIG. 7 shows the steps of the regeneration procedure of Oxalis tuberosa; and

FIG. 8 shows GUS staining of different stages of Oxalis tuberosa.

### DETAILED DESCRIPTION

Generally, the present disclosure provides a method for transforming tubers and a promoter that directs expression in tubers.

There is described herein a promoter comprising a nucleotide sequence which is SEQ ID NO:1 or a nucleotide sequence with 80% or greater identity to SEQ ID NO:1 which hybridizes to the complement of SEQ ID NO:1 under stringent conditions. The promoter may include a nucleotide sequence which is: a) SEQ ID NO:2; b) SEQ ID NO:3; or c) SEQ ID NO:4; or a nucleotide sequence with 80% or greater identity to SEQ ID NO:2, SEQ ID NO:3 or SEQ ID NO:4 which hybridizes to the complement of SEQ ID NO:2, SEQ ID NO:3 or SEQ ID NO:4. The nucleotide sequence of the promoter may have at least 85%, at least 90%, or at least 95% identity to SEQ ID NO. 1. Additionally, the promoter may be a nucleotide according to SEQ ID NO:2, SEQ ID NO:3, or SEQ ID NO:4, or a nucleotide sequence that is at least 85%, at least 90%, or at least 95% identity to SEQ ID NO. 2, SEQ ID NO:3 or SEQ ID NO:4.

There is further described a vector that includes the promoter described above. This vector may contain a target sequence operatively linked to the promoter. The target sequence may encode an enzyme of the class of orphan diseases for enzyme replacement therapy. The enzyme of a class of orphan disease can be adenosine deaminase, glucocerebrosidase, alpha-galactosidase, alpha-L-iduronidase, alpha-glucosidase, iduronate-2 sulphatase, arylsulphatase B, acid sphingomyelinase, or galactose-6-sulphatase. The encoded enzyme may also be human adenosine deaminase. The target sequence may also encode a peptide. For example, an antimicrobial peptide may be encoded. The target sequence may also encode a cytokine or a regulatory RNA.

In another aspect, there is described herein a cell transformed with the vector described above, or a plant comprising this cell. A vegetative propagule of the plant transformed with the vector and promoter described herein is also provided. Examples of propagules include a tuber or a stem tuber.

There is also described herein a method of producing a target protein in a tuber. This method includes transforming a cell with a vector which comprises the promoter described above, regenerating a fully functional plant; expressing the target protein; and isolating the target protein. The protein produced according to this method is also described.

There is further described a method for producing transformed Oxalis tuberosa. Oxalis tuberosa nodal stem explants are transformed with agrobacterium expressing an expression vector to form an infected nodal explant. Callus is removed from infected nodal explant and a bud is induced from the callus. A shoot is induced from the bud and a transformed Oxalis tuberosa is then produced from the shoot. The bud may be induced on the callus by incubating the callus in BI media. Additionally, the shoot bud may be induced from the callus by growing the bud in BI medium comprising gibberellic acid.

In another aspect, there is described herein a method for producing transformed Oxalis tuberosa comprising: infecting Oxalis tuberosa stem nodal segments with agrobacterium expressing an expression vector; inducing a morphogenic callus; isolating a transformed morphogenetic stem callus; inducing a shoot bud from the morphogenetic stem callus by growing the bud in a medium comprising gibberellic acid; germinating the bud; inducing a shoot from the bud; germinating and elongating the shoot; rooting the shoot in rooting media; and producing a transformed plant. The bud may be grown or induced in medium containing GA3 at an exemplary concentration of about 0.5 mg/L. Lower concentrations may also be used. The rooting media may comprise 0.1 mg/L naphthalene acetic acid (NAA).

There is further described herein a method for producing transformed Oxalis tuberosa comprising infecting Oxalis tuberosa nodal stem explant with agrobacterium expressing an expression vector, wherein the vector may comprise a promoter having a nucleotide sequence which is SEQ ID NO:1, SEQ ID NO:2, SEQ ID NO:3, or SEQ ID NO:4. The vector may include a target sequence operatively linked to the promoter. For example, the target sequence may encode an enzyme of a class of orphan disease, wherein the enzyme is adenosine deaminase, glucocerebrosidase, alpha-galactosidase, alpha-L-iduronidase, alpha-glucosidase, iduronate-2-sulphatase, arylsulphatase B, acid sphingomyelinase, or galactose-6-sulphatase. The target sequence may encode human adenosine deaminase. Other examples of target sequences include those that may encode a peptide, such as an antimicrobial peptide, a cytokine, or a regulatory RNA.

There is also described herein a plant produced by the methods described above and a vegetative propagule of such a plant.

One embodiment described herein is directed to an ocatin promoter isolated from a domesticated form of Oxalis tuberosa according to SEQ ID NO:1 or a nucleotide sequence with 80% or greater identity to SEQ ID NO:1 which hybridizes to the complement of SEQ ID NO:1 under stringent conditions (Maniatis et al., in Molecular Cloning (A Laboratory Manual), Cold Spring Harbour Laboratory, (1982) p 387 to 389. In this manual it is described that a labeled probe, consisting of the nucleotide sequence of interest, is incubated in hybridization solution comprising 6×SSC, 0.1M EDTA, 5×Denhardt's solution, 0.5% SDS and 100 mg/mL denatured salmon sperm DNA at 68° C. for 3-4 hours (100 ng/fragment DNA on a filter) or for 12-26 hours for 10 mg of DNA on a filter. The filter should be washed in a solution of 2×SSC and 0.5% SDS at room temperature. After 5 minutes, the filter can be incubated in a solution of 0.1×SSC and 0.5% SDS at 68° C. for 2 hours. Hybridizing nucleotides are then identified.

The specific sequences described herein, also include sequences that are “functionally equivalent” to the specific sequences noted above. Functionally equivalent sequences refer to sequences which although not identical provide the same or substantially the same function. Sequences that are functionally equivalent include any substitution, deletion, or addition within the sequence. Functionally equivalent sequences will also direct expression of exogenous genes to stem tubers.

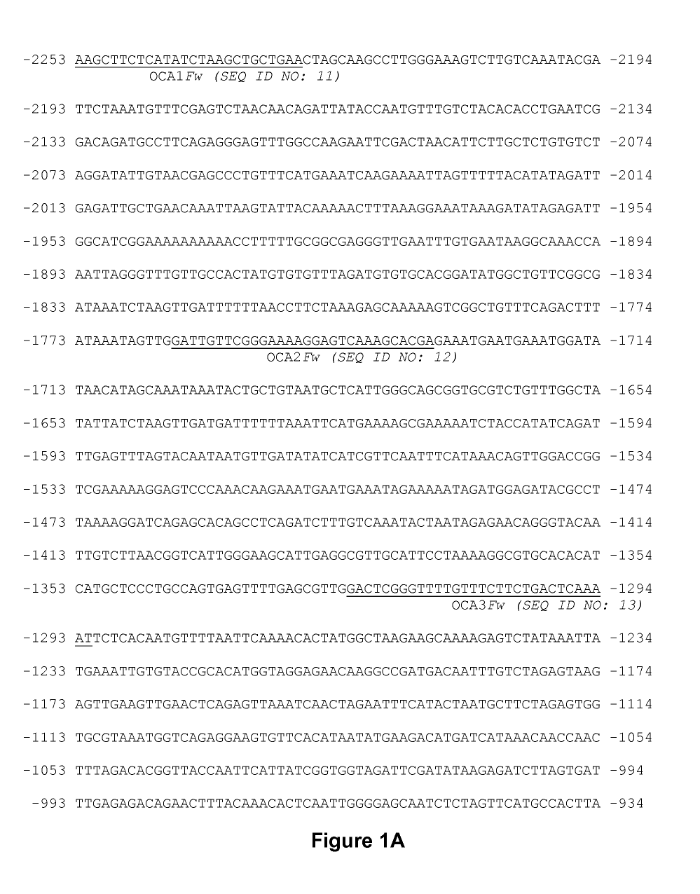

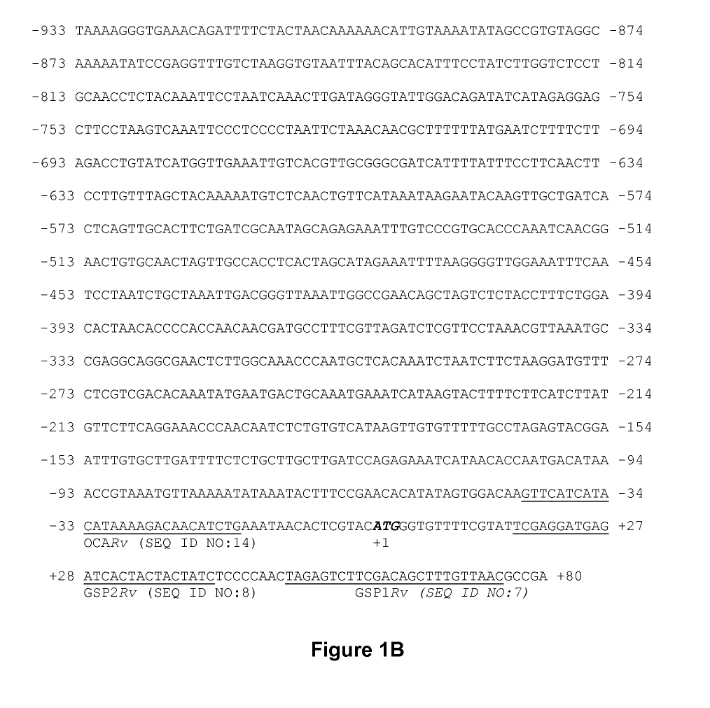

FIG. 1A and FIG. 1B shows the complete 5′ sequence of the Oca ocatin promoter. The promoter has 2253 base pairs and the nucleotides are numbered from the transcription start site (+1).

SEQ ID NO:1 refers to a nucleotide having the sequence of −16 bp to −76 bp according the numbering of FIG. 1A and FIG. 1B.

SEQ ID NO:2 refers to a nucleotide having the sequence of −16 bp to −1322 bp, and is referred to as pOC3.

SEQ ID NO:3 includes nucleotides −16 bp to −1762 bp and is referred to as pOC2.

SEQ ID NO:4 includes nucleotides −16 bp to −2253 bp and is referred to as pOC1.

SEQ ID NO:5 is a nucleotide comprising SEQ ID NO:1 and further comprising the nucleotides −15 to +1.

SEQ ID NO:6 is a sequence of the entire sequence shown in FIG. 1.

As used herein, the term “operatively linked” means that the components of the chimeric gene construct are positioned in such a way as to ensure proper transcription, or transcription and translation of the desired sequence.

A vector is described herein which comprised a target gene of interest wherein the target gene is operatively linked to an ocatin promoter. The target gene may encode any type of protein of commercial value or utility that can be expressed and possibly subsequently recovered from oca tubers. Examples of the types of protein products that can be so produced include: antimicrobial peptides, (AMPs), enzymes involved in starch production and metabolism, industrial enzymes of many kinds and proteins and peptides of medicinal or therapeutic value. Such enzymes or peptides of medical or therapeutic value may include: cytokines, (ie interleukin 4, 10, 35), hormones and growth factors, (ie insulin, glucagon-like protein −1, parathyroid hormone), orphan disease enzymes, (ie adenosine deaminase, glucocerebrosidase, alpha-galactosidase, alpha-L-iduronidase, alpha-glucosidase, iduronate-2-sulphatase, Arylsulphatase B, Acid sphingomyelinase, galactose-6-sulphatase) and enzymes such as lipases, peptidases, peroxidases and or any enzyme of commercial utility.

A protein is described herein that could be introduced to afford a plant protection or more specifically, tuber protection. This provides effective protection of plants while they are growing and protection of the harvested tubers from rotting during storage. For example, the anti-microbial peptides MsrA2 and temporin A have been shown to confer resistance in tobacco (Dmytro et al., 2007). The Oca once transformed with such peptides could be used directly or in a partly purified form as an animal feed without the need to completely purify the anti microbial peptides.

The promoter may also be useful to drive the expression of genes that would add to the agronomic performance of oca or other tubers such as genes that provide herbicide tolerance or protection against insect pests or fungal disease, or genes that provide growth advantages such as drought tolerance, or alter growth conditions in which a plant is able to grow. Additionally, the promoter may be used to express target genes that encode enzymes that alter the composition of the stored starch or other constituents of the tuber. It is possible to express an enzyme that makes different polysaccharides, ie hyaluronic acid or inulin. The complete or partial coding sequence of such genes may be in the sense or antisense orientation.

In a further embodiment the promoter may be useful to drive expression of genes that are tagged with an additional tag or sequence. Examples include but are not limited to affinity tags or epitope tags that are useful for isolating and purifying a protein of interest. The protein of interest can be fused with an affinity tag to aid in the subsequent recovery and purification of the target protein from the tubers. (see Terpe K., (2003). Overview of tag protein fusions from molecular and biochemical fundamentals to commercial systems. Appl. Microbiol. Biotechnol. 60:523-533.

Additionally the target gene may encode a protein that regulates DNA methylation.

In another embodiment the target gene may encode a regulatory RNA. A regulatory RNA is an RNA molecule that does not encode a protein, and can include but not be limited to, an RNA interference (RNAi), shRNA, or a microRNA (miRNA). Regulatory RNAs could be expressed to alter the storage characteristics of the tuber or alter the agronomic performance of oca or other tubers, for example.

The vector described herein may further comprise a 3′ untranslated region comprising a DNA segment that contains a polyadenylation signal and any other regulatory signals capable of effecting mRNA processing or gene expression. Examples of suitable 3′ regions are the 3′ untranslated region of the Agrobacterium Ti plasmid nopaline synthase gene (Nos gene) or the small sub-unit of the plant ribulose-5-phosphate carboxylase gene, (RUBISCO) or the 3′ untranslated region from the octatin gene, as described herein.

The vector construct described herein may also include enhancers, either translational or transcriptional enhancers as may be required.

The vector may further comprise a selectable marker gene. Selectable marker genes are well known in the art and include enzymes that provide antibiotic (i.e. kanamycin, hygromycin, spectinomycin) resistance or a visual colour change of cells and tissues and includes all genes that can help differentiate transformed and non-transformed cells. Examples of visual markers include the microbial beta-glucuronidase gene, (GUS) and the green fluorescent protein, (GFP) without being limited thereto.

The construction of vectors suitable for transformation of plants is known and routine to a person of skill in the art.

Also described herein are cells, vegetative propagules, and whole plants comprising a vector comprising the promoter. The plants may be Oxalis, commercially grown Oxalis tuberosa, naturally occurring feral forms of Oxalis tuberosa and related Oxalis species, potatoes, taro, true yams, sweet potato or any tuber-producing plant, without being limited thereto. Also described herein are plant cells and propagules of Oxalis transformed with the ocatin promoter of SEQ ID NO:1, SEQ ID NO:2, SEQ ID NO:3, or SEQ ID NO:4 commercially grown transgenic Oxalis tuberosa, naturally occurring feral forms of Oxalis tuberosa and related Oxalis species, potatoes, or any tuber-producing plant expressing the promoter of SEQ ID NO:1, SEQ ID NO:2, SEQ ID NO:3, or SEQ ID NO:4 without being limited thereto.

Also disclosed is a method of producing a target protein in a tuber by transforming a plant with an expression vector comprising a promoter disclosed herein. Common procedures can be used to extract proteins from complex mixtures of plant-derived materials. Generally separations are achieved by some form of chromatography wherein substances are separated based on size, net charge or hydrophobicity, (Ward et al., (2009) Protein purification, Current Analytical Chemistry 5(2): 1-21. If the protein of interest is fused to an affinity tag that has very specific binding properties to a ligand this can be used to efficiently capture the protein of interest on an affinity column, (Sharma et al. (2009), Plants as bioreactors: recent developments and emerging opportunities, Biotechnology Advances 27:811-832.

A further embodiment is a method for transforming Oxalis tuberosa and a method for introducing a vector comprising a promoter disclosed in this application into Oxalis tuberosa.

The above-described embodiments are intended to be examples only. Alterations, modifications and variations can be effected to the particular embodiments by those of skill in the art without departing from the scope, which is defined solely by the claims appended hereto.

The following examples are provided to illustrate the promoter and transformation method, but are not to be considered limiting. It is understood that modifications can be made.

### Example 1

### Isolation of the Oca (Oxalis tuberosa Mol.) Ocatin Gene Promoter

Ocatin is the most abundant storage protein of Oxalis tuberosa comprising 40-60% of the soluble protein. This protein, of molecular mass 18 Kd, is accumulated within cells of the pith and peridermis, (peel) of the underground stem tubers. Ocatin inhibits the growth of phytopathogenic bacteria and fungi and belongs to the Betv 1/PR-10/MLP protein family, (Flores et al., 2002).

Plant Materials and Growth Conditions.

Young leaves from tissue culture grown Oca (Oxalis tuberosa Mol.; 2n=8x=64) plants, maintained in growth chamber at 22° C. under fluorescent white light with 16/8 h light/dark cycle, were used for isolation of total genomic DNA.

Isolation of Genomic DNA.

Fifty mg samples of young oca leaf tissues were ground to a fine powder in liquid nitrogen. The powder was placed in 1.5-mL microtubes containing 700 μL 2% CTAB extraction buffer [20 mM EDTA, 0.1 M Tris-HCl pH 8.0, 1.4 M NaCl, 2% CTAB, plus 0.4% b-mercaptoethanol added just before use]. The solution was incubated at 65° C. for 40 min, gently mixed by inversion every 10 min; 500 μL of chloroform-isoamylalcohol (24:1) was added to the tubes and gently mixed for 1 min. Samples were centrifuged for 10 min. at 12,000 rpm; 600 μL of the supernatant was then transferred to a fresh tube followed by the addition of 500 μL chloroform-isoamylalcohol (24:1). This procedure was repeated twice. About 500 μL of the supernatant was then transferred to a fresh tube containing 700 μL of cold isopropanol (−20° C.). Samples were gently mixed by inversion and centrifuged at 12,000 rpm for 10 min, and hence it was possible to visualize the DNA adhered to the bottom of the tube. The liquid supernatant was decanted and the DNA pellet washed with 700 μL of 70% ethanol to eliminate salt residues adhering to the DNA, and then set to dry, at room temperature, with the tubes inverted over a filter paper, for approximately 12 h, or until the next day. Finally, the pellet was then re-suspended in 100 μL TE buffer (10 mM Tris-HCl pH 8.0, 1 mM EDTA pH 8.0) plus 5 μL ribonuclease (RNAse 10 mg mL-1) in each tube; this solution was incubated at 37° C. for 1 h, and subsequently stored at −20° C.

Isolation of the Ocatin promoter by GenomeWalker DNA Walking.

The 5′ flanking region of the Ocatin gene was isolated according to the instructions of the Universal GenomeWalker™ Kit (Clontech Laboratories, Palo Alto, Calif.) with some modifications. Isolated genomic DNA was digested with four restriction enzymes (DraI, EcoRV, PvuII, and StuI, respectively) to create blunt end fragments that were then ligated to a GenomeWalker adaptor to produce four GenomeWalker libraries.

FIG. 1 shows the 2.295 kb Ocatin promoter fragment isolated by two successive PCR based DNA walkings in GenomeWalker libraries. In FIG. 1, the nucleotides are numbered from the transcription start site (+1) and the positions of primers were underlined and labeled The gene specific primers were designed by using the Ocatin protein sequence (NCBI Genebank accession # AF333436). The primary PCR was performed with a gene-specific primer (GSP1 Rv; Table 1; FIG. 1) and the outer adaptor primer (AP1Fw) using the GeneAmp PCR System 2400 (PerkinElmer Applied Biosystems). The amplifications began with 5 cycles of 94° C. for 25 s and 72° C. for 3 min, followed by 30 cycles of 94° C. for 25 s and 68° C. for 3 min, and a final extension at 68° C. for 7 min. For the second round of genome walking, the diluted primary PCR products served as the template for the secondary ‘nested’ PCR with a nested gene specific primer (GSP2Rv) and the nested adaptor primer (AP2Fw). The secondary PCR products were analyzed by agarose gel electrophoresis. The major bands were purified from gels with a QIAEXII Gel Extraction Kit (Qiagen), cloned into pGEMT Easy Vector (Promega, Madison, Wis.) and sequenced by automated nucleotide sequencing at NRC-PBI (National Research Council of Canada—Plant Biotechnology Institute, Saskatoon).

TABLE 1
Oligonucleotide Primers Used to Amplify
5′ Flanking Regions of Ocatin Gene

| Primer | |
|---|---|
| Name | Sequence |
| GSP1 Rv | 5′ GTTAACAAAGCTGTCGAAGACTCTA 3′ |
| GSP2 Rv | 5′ GATAGTAGTAGTGATCTCATCCTCGA 3′ |
| AP1 Fw | 5′ GTAATACGACTCACTATAGGGC 3′ |
| AP2 Fw | 5′ ACTATAGGGCACGCGTGGT 3′ |
| OCA1 Fw | 5′ AAGCTTCTCATATCTAAGCTGCTGAAC 3′ |
| | HindIII |
| OCA2 Fw | 5′ AAGCTTGATTGTTCGGGAAAAGGAGTCAAAGCACGA 3′ |
| | HindIII |
| OCA3 Fw | 5′ AAGCTTGACTCGGGTTTTGTTTCTTCTGACTCAAAAT 3′ |
| | HindIII |
| OCA Rv | 5′ GGATCCAGATGTTGTCTTTTATGTATGATGAAC 3′ |
| | BamHI |

Table 2 lists the primers and template DNA used for generating entry clones.

TABLE 2
Primers and Template DNA used for Generating Entry Clones

| Amplified DNA | | Template DNA |
|---|---|---|
| fragments with | | (Plasmid pBluskript II KS(+) |
| specific recombines site | Pair of primers | carries following DNA sequence) |
| attB4-OC2-attB1 | Oc2B4Fw | OC2 Tuber specific promoter |
| (Size 1747 bp) | 5′GGGGACAACTTTGTATAGA | from Oca (pOC2-GUS was used |
| | AAAGTTGGATTGTTCGGGAAA | as a template) |
| | AGGAGTCAAAGCACGA-3′ | |
| | Oc2B1Rv | |
| | 5′GGGGACTGCTTTTTTGTAC | |
| | AAACTTGCAGATGTTGTCTTT | |
| | TATGTATGATGAAC 3′ | |
| attB1-PinII-attB2 | PinIIB1Fw | PinII (Potato Proteinase inhibitor |
| (size 279 bp) | 5′GGGGACAAGTTTGTACAAA | II) apoplast specific signal peptide |
| | AAAGCAGGCTATTCACAGACA | (Gene Bank Accession # X04118; |
| | CTCTTCACCCCAA 3′ | A 279 bp PinII DNA sequence was |
| | PinIIB2Rv | synthesized at NRC-PBI, DNA |
| | 5′GGGGACCACTTTGTACAAG | sequencing facility) |
| | AAAGCTGGGTAAGCCTTCGCA | |
| | TCAACATGCTCCAT 3′ | |
| attB2-hADA-attB3 | hADAB2Fw | ORF of hADA gene (human Adenosine |
| (size 1089 bp) | 5′GGGGACAGCTTTCTTGTAC | Deaminase gene) (Gene Bank |
| | AAAGTGGTGCCTAGAATGGCT | Accession # CAH73885.1; A 1089 bp |
| | CAAACTCCTGCTTTTGAT 3′ | artificial ORF was synthesized at |
| | hADAB3Rv | NRC-PBI, DNA sequencing facility) |
| | 5′GGGGACCACTTTGTACAAG | |
| | AAAGCTGGGTTTATTAAAGAT | |
| | TTTGACCAGCAGA 3′ | |

### Example 2

### Construction of Transformation Vectors

Construction of Chimeric Reporter Gene Fusions.

The Ocatin putative promoter deletion derivatives were operatively linked with the β glucuronidase (GUS) reporter gene by cloning the various 5′ deletion fragments into the polylinker region of the binary vector pBI101 (Clontech Laboratories) upstream of a promoterless GUS gene cassette.

The 2.238 kb (pOC1), 1.747 kb (pOC2) and 1.307 kb (pOC3) promoter fragments were produced by PCR amplification with Vent® DNA Polymerase (New England Biolabs, Beverly, Mass.) using forward primers OCA1 Fw, OCA2Fw and OCA3Fw (with a HindIII site), respectively, and a common reverse primer OCARv (with a BamHI site). The resulting products were digested with HindIII and BamHI; and further subcloned into the pBI101 to form pOC1-GUS, pOC2-GUS and pOC3-GUS, respectively. These three vectors were used in a transient expression study.

All of the promoter constructs were sequenced to identify any possible PCR-introduced mutations.

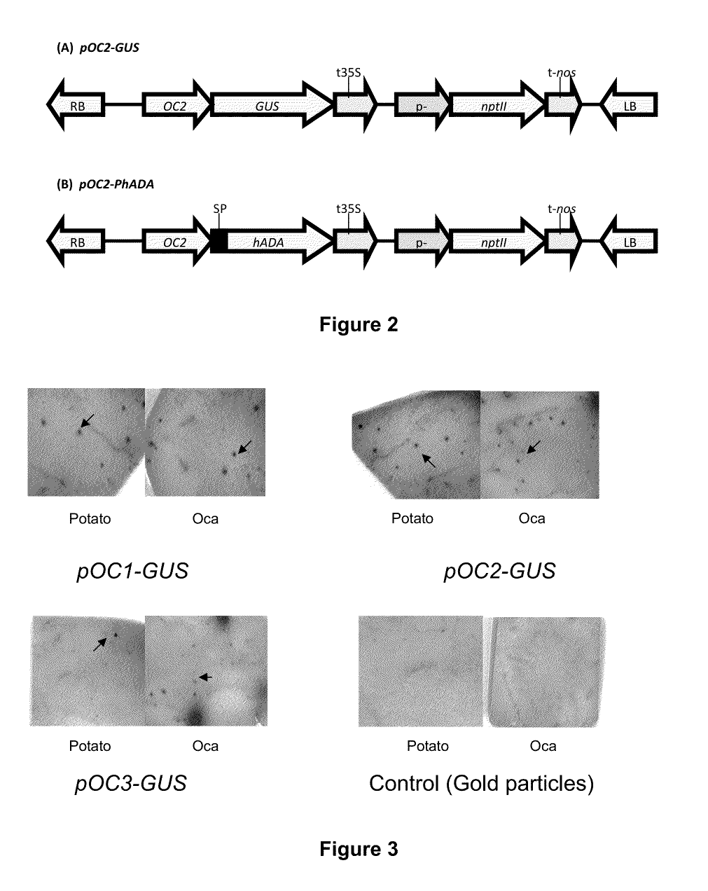

FIG. 2 is a schematic diagram of the constructs used for Oca transformation to generate plants expressing either GUS or human ADA in tubers. (A) pOC2-GUS: The construct contains a GUS gene under the control of the OC2 tuber specific promoter and terminator (t35S). (B) pOC2-PhADA: This construct contains a gene encoding the human Adenosine deaminase enzyme under the control of the OC2 tuber specific promoter followed by the apoplast specific Pin II signal peptide (SP) and terminator (t35S). The marker gene neomycin phosphotransferase II (nptII), regulated by the Nopaline synthase promoter (p-nos) and terminator (t-nos), was integrated into the construct (RB, right border of the T-DNA; LB, left border of the T-DNA).

### Example 3

### Transient GUS Expression Assays in Potato and Oca

DNA-coated microparticles were prepared using the CaCl₂/spermidine method as described by Sanford et al. (1993). A 1-μg aliquot of the each pOC1-GUS, pOC2-GUS and pOC3-GUS plasmid was mixed with 1 mg of gold particles (1.0 Micron Gold, Bio-Rad) in the presence of 1M spermidine and 16 mM CaCl₂. Potato (Solanum tuberosum L. cv. Desiree) and Oca tubers were transversely cut to a thickness of 3 mm. Leaves; stem and tuber discs tissues were placed on wet 3 mm filter paper in Petri dishes and incubated at 4° C. for 4 h prior to bombardment. A biolistic gun device (PDS-1000/He; Bio-Rad) was used to deliver the plasmid-coated gold particles (1 mg per bombardment) and uncoated gold particles (negative control) using the following parameters: the stopping screen was positioned 3 cm below the rupture disk; the target tissue was positioned 9 cm below the stopping screen; and the helium pressure was 1350 psi. The bombarded tissues were then incubated for 18 h at 25° C. in the dark.

The next day, histochemical assays of GUS activity were performed according to Jefferson (1987) using 5-bromo-4-chloro-3-indolyl P-D-glucuronide (X-gluc; Biosynth., Staad, Switzerland). Plant materials were placed in 2 mM X-gluc, and incubated overnight at 37° C. in the dark. After staining was completed, plant tissues were cleared with ethanol at room temperature. Tissues were examined using a stereo microscope for the detection of blue spots. Such blue spots are depicted herein in the figures as dark spots, or intensely shaded regions.

FIG. 3 shows transient expression of the GUS enzyme after bombardment with either pOC1-GUS, pOC2-GUS, pOC3-GUS. The top left panel shows GUS expression driven by the 2.238 kb (pOC1) promoter in potato and in Oca, as seen as dark spots identified by the arrows. The top right panel shows GUS expression from the 1.747 kb (pOC2) promoter in potato and Oca, again identified by blue spots (shown in the instant figure as dark spots). The bottom left panel shows the expression of GUS in potato and Oca driven by the 1.307 kb (pOC3) promoter. A control is shown in the bottom right panel, in which potato and Oca were bombarded with uncoated gold particles and no GUS expression was seen. FIG. 3 demonstrates the typical GUS staining pattern for plants containing the different constructs. While the pattern of expression for a given construct was generally consistent, the amount of expression varied.

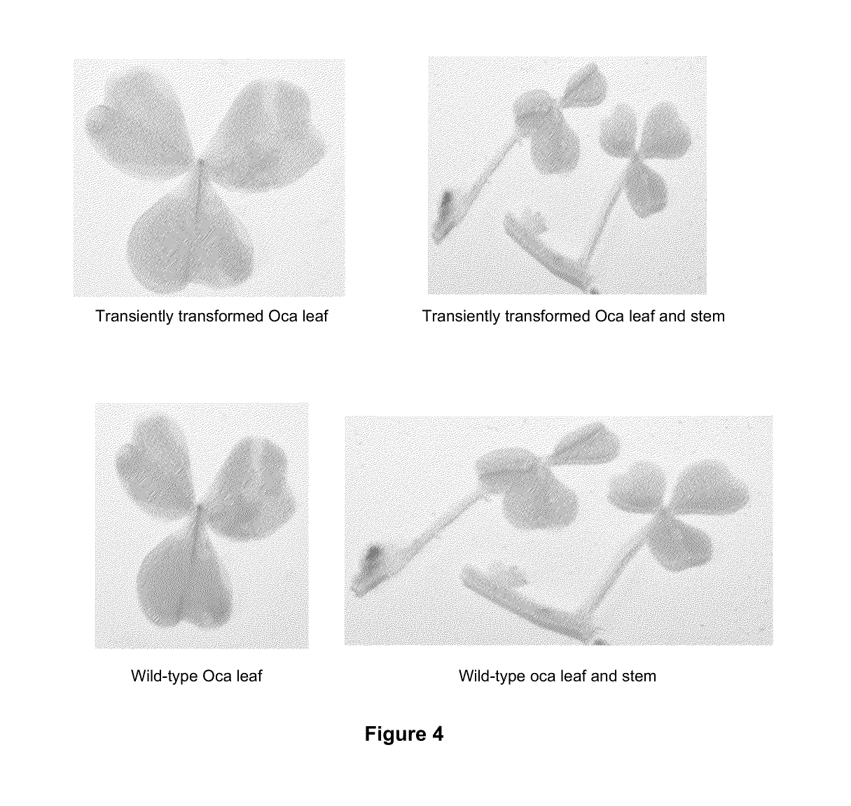

FIG. 4 demonstrates that Oca leaves and stems transiently transformed as described above do not express GUS suggesting that this promoter has little or no activity in leaves or stems. The GUS gene was not transiently expressed in Oca leaf and stem when bombarded with any of pOC1-GUS, pOC2-GUS or pOC3-GUS vectors.

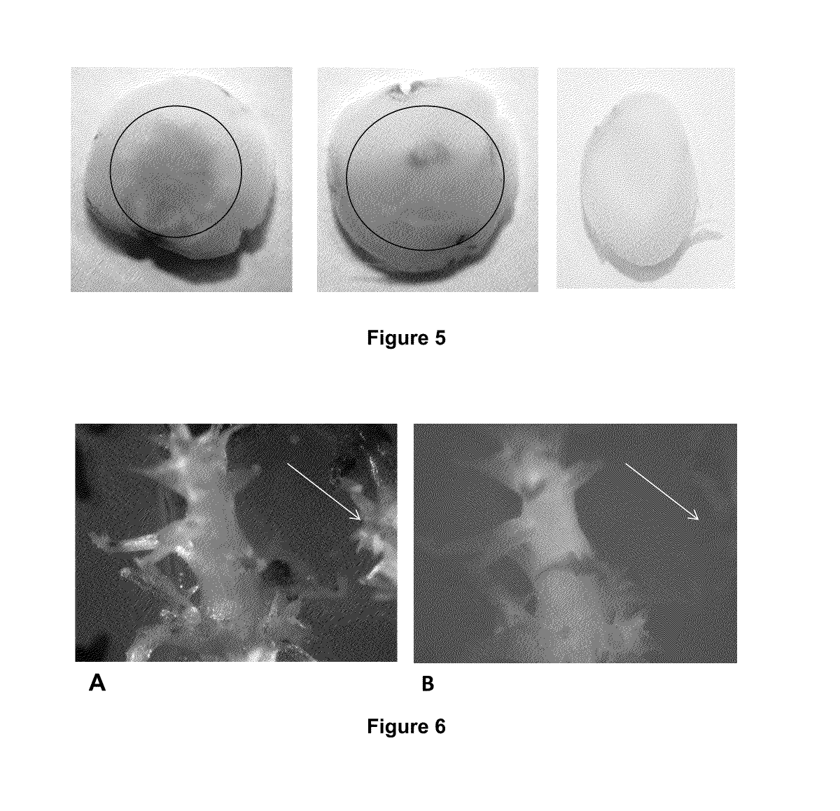

FIG. 5 shows transient expression of GUS using pOC2 in Oca. The left panel is a bottom view of an Oca tuber cross section showing GUS expression in the pith, or cortex (see circle). The middle panel is a top view of a tuber cross section showing GUS expression in the epidermis layer (outer skin). The right panel is a control showing a control Oca tuber as reference. No GUS staining is seen in the control Oca tubers.

Table 3 summarizes the levels of GUS expression in potato and Oca transiently transformed with the pOC1-GUS, pOC2-GUS and pOC3-GUS vectors. The highest levels of expression were seen using pOC2-GUS.

TABLE 3
The effect of different vectors on transient
GUS expression in Potato and Oca tissues.

| | Average GUS Spots ± Standard Error | | | |
|---|---|---|---|---|
| | tissue | | | |
| Vectors | Potato Tuber | Oca Tuber | Oca Leaf | Oca Stem |
| pOC1-GUS | 10.7 ± 2.37 | 11.7 ± 2.25 | 0 | 0 |
| pOC2-GUS | 15.7 ± 2.39 | 21.0 ± 2.80 | 0 | 0 |
| pOC3-GUS | 7.3 ± 1.85 | 12.7 ± 2.28 | 0 | 0 |
| Control | 0 | 0 | 0 | 0 |
| (Gold Particle) | | | | |

### Example 4

### Construction of Vectors for Stable Transformation in Oca

On the basis of transient GUS expression results, pOC2-GUS was used in stable transformation experiments. A further expression vector, pOC2-PhADA, was constructed to produce the human Adenosine deaminase enzyme targeted to the apoplast by using the PinII signal peptide (Liu et al. 2004). Both vectors (shown in FIG. 2) contained the 1.747 kb OC2 tuber specific promoter fragment. The amino acid sequence of hADA (GenBank: CAH73885.1) was back-translated using plant preferred codons, and the resulting artificial ORF was synthesized at NRC-PBI, DNA sequencing facility. By using a specific pair of primers and template DNA, three kinds of DNA fragments (attB4-OC2-attB1, attB1-PinII-attB2 and attB2-hADA-attB3) were amplified by polymerase chain reaction (PCR) (Table 2). The integrity of these fragments was verified by automated nucleotide sequencing at NRC-PBI (National Research Council of Canada—Plant Biotechnology Institute, Saskatoon). The entry clones were obtained by BP clonase reaction between said three DNA fragments and specific donor clones from Gateway (Invitrogen; Catalogue #12537-023). The vector pER598 was derived from pKm43GW (Karimi et al. 2005) and used as a destination clone. A binary vector pOC2-PhADA was generated by inserting a single gene cassette into pER598 using LR clonase reactions to transfer the gene cassette from the entry clone to the destination vector, essentially according to the protocol of the MultiSite Gateway Three Fragment Vector Construction Kit (Invitrogen; Catalogue #12537-023). Both of the vectors were electroporated into disarmed Agrobacterium tumefaciens strain GV3101-pMP90 (Koncz and Schell, 1986) prior to plant transformation experiments.

### Example 5

### Comparative Attempts to Develop an Oxalis tuberosa Transformation Procedure Via Direct Shoot Regeneration

There are a variety of strategies used to achieve regeneration in transgenic and non-transgenic plants, some of them are direct and other methods are indirect. The direct generation of a somatic embryo from a primary transformed cell is a desirable way of achieving regeneration because the embryo is a bipolar structure with shoot and root meristems developing simultaneously and thus the additional step of rooting of regenerated shoots is not needed. Another reason why embryogenesis is a preferred route to regeneration is because of the single-cell origin of the embryo which potentially increases transformation efficiency and eliminates the possibility of generating chimeras. Chimeric shoots may be comprised of a mixture of transformed and non-transformed cells because of the multiple cell origin of these structures.

Conventional protocols for transformation which focus on direct shoot regeneration were not successful for Oca as none of the shoots produced were stably transformed.

FIG. 6 shows the expression of GFP in Oxalis tuberosa shoots 4 weeks after the initial transformation. Panel A shows a picture of the shoots taken under visual light, while panel B shows GFP fluorescence. The arrows in the panels indicate the non-transformed shoots. The expression of the transgene disappeared, however, as the shoots developed further. It was discovered however that cells that were permanently transformed comprised undifferentiated, relatively slow-growing callus that developed on cut explants surfaces. This will be described further in Example 6.

Typically, the optimal strategy for development of a transformation system for any species is to find an initial explant with high morphogenic capacity which comprises at least some fast dividing meristematic cells. This approach was attempted with Oca as described below.

Source of Plant Explants.

Young shoots from greenhouse-grown Oca plants were surface sterilized for 30 sec in 70% ethanol, followed by immersion in 10% commercial bleach for ten minutes. Explants were rinsed with sterile ddH₂O, cut to segments with 1-2 buds which were placed in Magenta™ jars inserted into media comprised of Gamborg B5 salts and vitamins, 3% sucrose and solidified with 0.8% agar. Typically, 4 cuttings were placed in each Magenta™ jar and were grown with a 16 h photoperiod in full light at 24° C. After 4 to 5 weeks shoots induced from axillary buds of the initial stem segments reached up to 7-8 cm in length. These shoots were used as a source of explants in all tissue culture and transformation experiments. Shoots were re-cut and transferred to fresh medium every 8-10 weeks to maintain a source of explant material.

Development of the Initial Explant and Shoot-Inducing (SI) Media.

To determine the initial explant with the highest morphogenic potential in Oxalis tuberosa different parts of the stem were tested over a wide range of culture media compositions containing all commonly used plant growth regulators. It was established that the most responsive tissue was the axillary bud meristem. Each Oca stem node has a pre-existing axillary shoot bud meristem. When such bud meristems and adjacent nodal areas are wounded or cut through, multiple shoots can be induced from a single node on shoot-inducing medium. It was established that only a radial cut or partial cut (wounding) in the radial direction was efficient for shoot induction.

Optimal medium composition for shoot induction was determined to comprise Gamborg B5 salts and vitamins, 3% sucrose and both the auxin 2,4D and the cytokinin TDZ in concentration 5 μM for 2,4D and 2.5 μM for TDZ, respectively.

Thus, explants comprising nodal segments were cut (or wounded) radially and placed on this optimal shoot-inducing (SI) medium. Shoot development was initiated as early as 1 week after cultivation and after 4-5 weeks of cultivation elongated shoots were present on the majority of explants. During the first 2 weeks of cultivation explants were kept in the dark, at 23° C. Further cultivation was continued under full light with a 16 h photoperiod GFP shoots were maintained in partial darkness to avoid photo-bleaching (NL please expand).

Transformation Vectors and Agrobacterium Culture Preparation.

The following Agrobacterium vectors were constructed for stable transformation of oca and electroporated into disarmed Agrobacterium tumefaciens strain GV3101-pMP90 (Koncz and Schell 1986).

p35SGFP:

This vector comprises the green fluorescent protein (GFP) gene, driven by a constitutive promoter (CaMV 35 S) and a 35S terminator. The nptII gene with a NOS promoter and terminator (for kanamycin resistance) was also included for selection of transformed cells.

POC2PhADA:

This vector was constructed with the human adenosine deaminase (hADA) gene (GenBank: CAH73885.1; synthesized at NRC/PBI for this construct) driven by a tuber specific (OC2) promoter with a potato proteinase inhibitor II (PinII) apoplast specific signal peptide (Gene Bank Accession # X04118) and a 35S terminator. The nptII gene with a NOS promoter and terminator, providing kanamycin resistance was included, for selection of transformed cells.

pPBI3010:

This vector contains a fusion gene (gus::nptII) conferring both β-glucuronidase (GUS) and neomycin phosphotransferase (nptII) functions with a constitutive 35S35SAMV promoter, a NOS (nopaline synthase) terminator and an intron. The construct (pPBI3010) was electroporated into the disarmed Agrobacterium strain EHA105 which was coded LBG 66.

Agrobacterium Culture Preparation.

Agrobacteria for transformation experiments were cultured overnight in 2YT medium with antibiotics (50 mg/l each of rifampicin, gentamycin and spectinomycin for p35SGFP and POC2PhADA; and 30 mg/l rifampicin, 25 mg/l gentamycin for LBG-66) were harvested by brief centrifugation and resuspension in fresh 2YT media without antibiotics. For co-cultivation, the Agrobacterium suspension was diluted to a final OD of 0.05 at A₆₆₀.

Transformation Procedure.

The initial explants comprised stem nodal segments from sterile microclonal plants with leaf excised with the basal part of petiole still attached. The explant axillary bud and adjacent meristematic tissue, were wounded by cutting with a scalpel while positioned in the Agrobacterial suspension. Explants were left in the Agrobacterium culture for and additional 2 hours, blotted dry on filter paper and placed on shoot inducing (SI) media without antibiotics. Explants were placed in the dark at 22° C. for 3 days.

After 3 days of co-cultivation explants were washed in sterile ddH₂O to remove excess Agrobacteria, blotted dry on filter paper and transferred to the same medium with antibiotics (kanamycin 50 mg/L and timentin 200 mg/L). Explants, 7 explants per plate, were cultivated in deep Petri dishes. Plates were sealed with surgical tape.

Transgenic Shoot Analysis.

p35SGFP:

After 4 weeks of cultivation elongated shoots developed on the most of the treated explants. Intact shoots were checked for GFP expression under fluorescence. A total 6 plates with 7 explants each were tested. GFP expression was detected in about 70% of the shoots which developed from the explants. In some instances the same explant generated shoots with and without GFP expression. Non-transformed Oca shoots were used as a control. No green fluorescence was detected in control plates. Shoots were left for further development on the same plates for an additional 4 weeks in partial light. These shoots were re-tested for GFP expression but no GFP fluorescence was detected.

POC2PhADA:

After 5 weeks of cultivation 16 large elongated shoots from 5 different explants were recovered from initial explants and transferred to the hormone free (HF) media with kanamycin 50 mg/L and 200 mg/L timentin for further development. Tissue from each shoot was collected and tested for presence of hADA by PCR analysis. 15 of 16 shoots tested positive for presence of the hADA gene. The same shoots were re-tested again by leaf PCR after an additional 4 weeks of cultivation. None of the shoots tested positive for hADA at the time.

LBG 66:

After 4 weeks of cultivation 36 initial explants together with developing shoots were stained for GUS activity. All explants expressed GUS activity, with the strongest expression in wounded areas of the initial explants. In 25 explants GUS expression was also detected in the basal part of developing shoots. Only 12 explants had shoots with GUS expression in upper parts of the developing stem. Prior to staining for GUS activity, 10 shoots from different explants were excised and transferred to the hormone-free media with antibiotics (50 mg/L kanamycin and 200 mg/L timentin) for further cultivation. These shoots were tested for GUS activity after an additional 4 weeks of cultivation and none of them was positive.

In all transformation experiments where regeneration by direct morphogenesis was observed, only shoots with transient expression were regenerated. One hundred percent of the initial explants transformed with the GUS LBG 66 vector tested positive after 4 weeks of cultivation, indicating that oca tissue is highly receptive to transformation by Agrobacterium tumefaciens but the transformed cells did not regenerate stably transformed shoots. It was observed however that a callus developed on cut/wounded sites. Samples of this callus were transferred to the SI media with antibiotics and over time, very slow growing callus cultures were established. Portions of this callus were tested for GUS activity at 6 week intervals for a period of over a year. Calluses consistently showed very high levels of GUS expression. This indicates that stable transformation was only achieved in dedifferentiated callus cells that subsequently failed to develop into shoots on the shoot induction medium. This example illustrates the difficulties experienced in attempting to create a stable transformation system for Oca based on conventional direct shoot morphogenesis.

### Example 6

### Development of an Oxalis tuberosa Transformation Procedure Via De Novo Shoot Formation from Stably Transformed Morphogenic Callus

Regeneration Procedure.

Regeneration protocols for de novo shoot formation from dedifferentiated cells such as callus typically include several steps where each step has individual requirements of medium composition as well as specific cultivation conditions. For Oca such steps include: 1) morphogenic callus induction 2) bud induction from callus 3) bud germination or shoot induction 4) shoot germination/elongation and 5) rooting.

Callus Induction from Initial Explants

Morphogenic callus was induced from radially cut stem nodal portions of sterile microclonal plantlets. Each node was cut in to two 2 mm thick slices. Experiments were conducted to optimize a callus-inducing medium (CI) which was determined to comprise Gamborg B5 salts and vitamins, 3% sucrose, 0.75% agar that was supplemented with the synthetic auxin picloram, at concentration 1 μM, and the cytokinin TDZ at a concentration 2.5 μM. Explants were placed on media in deep Petri dishes, wrapped with surgical tape and cultivated in the dark at 23-24° C. After approximately 5-6 weeks of cultivation a yellowish compact callus developed on the upper side of the explants.

Bud Induction from Callus (BI)

After 5-6 weeks, calluses reach an average size of 1 cm in diameter. The initial explant portion was carefully surgically separated from the callus and calluses were transferred to a bud-inducing media (BI). BI media has the same basic composition as CI medium except the growth regulator component required was determined to be entirely different. In BI media, surprisingly, the only growth regulator needed is gibberellic acid GA₃, with an optimal concentration of 0.5 mg/L. Calluses, 3-4 per plate, were placed in deep Petri dishes, wrapped with surgical tape and cultivated in the dark at 23-24° C. The first indication of the development of buds can be as early as 3 weeks after transfer to the BI medium. However, in some calluses bud development was only evident after 5 to 6 weeks. Developing buds are reddish in color and appear as very small bumpy structures on the callus surface. It may be necessary to transfer calluses to fresh BI media for a second round of growth to induce more buds from the same callus.

Bud Germination or Shoot Induction (SI)

The first buds appeared as bumpy pink-to-red structures on the surface of the callus but have the same texture as callus. At later stages of development buds are well-structured and have a distinct shiny smooth surface. There are two types of bud structures: 1) bud-like, which are of light pink color; and 2) tuber-like, which are of a dark red color. For further development these structures were transferred to fresh SI medium as above but without growth regulators. Explants are cultivating on SI media in the deep Petri dishes, wrapped with surgical tape in the dark at 23-24° C. Typically, more than one transfer to growth regulator free medium is needed before development of buds into shoots was achieved. Usually, this step takes 6-10 weeks. At this stage it is very important to keep bud structures and underlying callus intact and not to cut it when transferring to fresh medium.

Shoot Germination/Shoot Elongation (SE)

Typically buds/shoots in oca cultures developing in clusters and for further development individual shoots need to be separated from such clusters. Once buds start elongation, it is safe to separate them from callus and from each other. The optimal shoot elongation media (SE) is the same medium as SI described above but supplemented with the cytokinin zeatin at concentration 0.1 μM. While the addition of zeatin is not required, its presence promotes shoot development and elongation. At this stage more frequent transfers to the fresh medium (2-3 weeks intervals) are required to stimulate fast shoot growth. Once shoots became elongated, they should be transferred to light.

Rooting

Excised Oca shoots root easily on hormone-free media, however, the addition of low concentrations of naphthalene acetic acid (NAA) at about 0.1 mg/L improves the process. Once shoots reach 1.5-2 cm in length they can be transferred to Magenta™ jars to accommodate growth. During this step shoots are grown in light with a 16 h photoperiod, at a temperature of 23° C. Once shoots are big enough (about 4 cm), they can be successfully transferred to soil.

Transformation Vectors and Agrobacterium Culture Preparation.

Once the procedure to regenerate shoots from morphogenic callus was developed, the LBG-66 Agrobacterium strain was used to develop a full transformation procedure based on regeneration by de novo shoot induction from morphogenic callus. Agrobacterium culture preparation was the same as described above.

Stem segments of microclonal plantlets were cut and leaves removed. Only nodal segments were used. Nodal segments of stem were radially cut under a binocular microscope with a scalpel and dipped into the Agrobacterium suspension. Pre-existing axilary buds were surgically removed. Radially cut segments about 2 mm thick were transferred to the Agrobacterium suspension.

Cut explants segments were incubated in the Agrobacterium suspension for 2 hours. Subsequently explants were briefly blotted on sterile filter paper to remove the excess Agro suspension before transferring to the co-cultivation media (the same as CI media but omitting antibiotics). Plates were wrapped with surgical tape and placed in the dark at room temperature (22-23° C.) for co-cultivation (4 days).

Transgenic Shoot Regeneration

Shoot regeneration involved the following steps of callus induction, bud induction from the callus, and bud germination leading to shoot induction and elongation.

Morphogenic Callus Induction

After 4 days of co-cultivation explants were washed in sterile ddH₂O, blotted dry on filter paper and then transferred to the CI media supplemented with both kanamycin and timentin (100 mg/L kanamycin and 200 mg/L timentin). Five explants were placed on each plate (deep Petri dishes); plates were wrapped with surgical tape and cultivated in the dark at 23° C. Explants were cultivated on the CI media for at least 4 weeks or until callus reached a size of 1 cm in diameter. In some cases a 2^{nd} transfer to the CI media is required to recover a significant volume of callus.

Bud Induction from Morphogenic Callus

Developed calluses were excised and transferred to the BI media with antibiotics (kanamycin 100 mg/L and timentin 200 mg/L). At this stage 4 explants were cultivated per plate. Again, plates were cultivated in the dark at 23° C. Typically after 5-6 weeks buds developed.

Bud Germination/Shoot Induction

Explants with developed buds were transferred to SI media with antibiotics. At this stage the concentration of kanamycin was decreased to 50 mg/L and timentin concentration was kept at the same level (200 mg/L). Explants were cultivated on SI media until bud development progressed. Plates were maintained as in the previous steps in the dark, at 23° C.

Shoot Elongation

Explants with germinating buds were transferred to the SE media with the same level of antibiotics as SI media (see above).

Rooting

Elongated shoots were transferred to the rooting media with kanamycin 25 mg/L and timentin 200 mg/L in Magenta™ jars (5 shoots per jar) and cultivated in the light with a 16 h photoperiod, and a temperature of 23° C.

Transfer of Transgenic Plants to Soil and Transgenic Tuber Production

After rooted shoots reached at least 4 cm they were transferred to soil and grown under a 16 h photoperiod. To induce tuber production plants were transferred to a 12 h photoperiod and after 4-5 weeks tubers development was observed.

Transgenic Shoots/Tuber Analysis.

To confirm the transgenic nature of explants/developed shoots and for estimating transformation efficiency of the established procedures tissue samples were tested for GUS expression at each step of the regeneration process. First, portions of calluses from all explants transferred to the BI media were stained for GUS activity. It appeared that over 90% of all calluses expressed the GUS gene. After this stage samples were taken randomly after each transfer and all tested explants were positive for GUS expression. Additionally, the transgenic nature of explants was confirmed by the ability to grow on kanamycin at a concentration of 100 mg/L. Control explants (non-transformed Oca stem segments) were not able to produce any callus at this level of kanamycin and died after 2 weeks of cultivation on this concentration of kanamycin. Tubers, produced by transgenic plants also tested positive for GUS expression. Transgenic tubers produced by the first generation of transgenic plants were harvested and re-planted. GUS expression was confirmed up to the 3^{rd} generation of tubers.

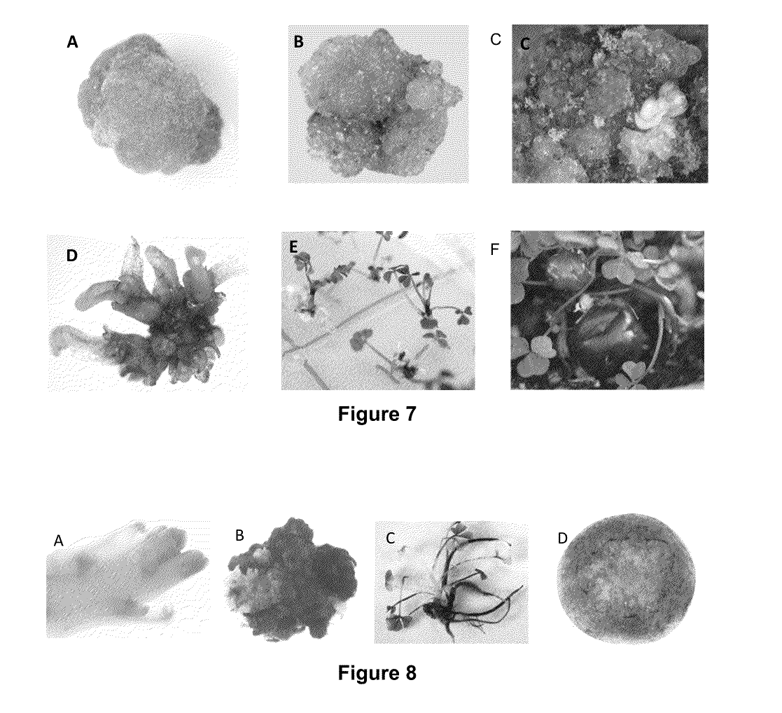

FIG. 7 outlines the steps of the regeneration procedure of Oxalis tuberosa. Panel A shows a morphogenic callus developed after 5 weeks on CI media. Panel B shows buds that developed after 6 weeks on BI media. Panel C shows a bud germinating on SI media. Panel D shows shoots growing on SE media. Shoots on rooting media are show in Panel E, while tubers are shown in Panel F.

FIG. 8 shows GUS staining at various stages of transgenic Oxalis tuberosa regeneration from morphogenic callus (transformed with LBG 66). Panel A shows control, non-transformed shoots from tissue culture. Panel B shows callus with buds and germinating shoots. Panel C shows a rooted plant and panel D shows a radially cut tuber. GUS staining is evidenced by the staining in panels B, C and D, compared with the non-staining control in panel A.

### Example 7

### Partial Purification of hADA from Transgenic Oca Tubers

Demonstration that transformed Oca tubers can serve as a production platform for a novel commercially valuable human protein was shown by transformation with the POC2PHADA vector by the method described in Example 6. In order to enhance detection of hADA the enzyme was partially purified as described below.

Crude protein was extracted from approximately two grams of tuber tissue. Both transgenic and non-transformed Oca control tuber samples were homogenised with a mortar and pestle in protein extraction buffer (50 mM Tris-HCl, pH 7.0 and 20 μl of β-mercaptoethanol in 100 ml buffer) at a ratio of 6 ml buffer per gram tuber tissue with a pinch of PVPP (polyvinyl polypyrrolidone). After centrifugation (12,000 rpm, 25 min) at 4° C., the supernatant was collected and fractionated by salting out with increasing concentrations of ammonium sulfate. Solid ammonium sulfate was slowly added to the above crude extract with gentle stirring, up to 35% saturation in 10 minutes. The protein was precipitated by centrifugation (12,000 rpm, 25 min) at 4° C. The supernatant was decanted to another beaker, and solid ammonium sulfate was added to 70% saturation. This mixture was treated as above, another protein precipitate was obtained at 35%-70% saturation of ammonium sulfate. The protein pellet was suspended in 1 ml of ice-cold protein extraction buffer and filtered using a 0.2 micron syringe filter. Further desalting of the protein sample was accomplished by using pre-packed column (Bio-Rad, Econo-Pac 10 DG Disposable chromatography column, 10 ml column, Cat #732-2010) resulting in eluted desalted total protein in 4 ml of protein extraction buffer. Protein concentrations were determined with a Bio-Rad protein assay kit following the manufacturer's instructions (Bio-Rad Laboratories Inc., Hercules, Calif.).

The presence of the hADA gene was determined via the enzymatic activity of the hADA protein. Each sample was evaluated for enzyme activity (U/L) by using a Bio-Quant adenosine deaminase assay kit (Bio-Quant; Cat # BQ 014-EALD). The results of these assays are shown in Table 4. Transformed tubers showed 13.5 U/L ADA activity while control untransformed Oca tubers showed 0.0 U/L ADA activity.

TABLE 4
Expression of hADA

| | | Protein content | Sample used for | ADA activity* |
|---|---|---|---|---|
| No | Sample | (mg/ml) | ADA assay (ml) | (U/L) |
| 1 | Transgenic | 0.28 | 0.5 | 13.5 |
| | oca tuber | | | |
| 2 | Wild type | 0.29 | 0.5 | 0.0 |
| | oca tuber | | | |

*One unit of ADA is defined as the amount of ADA that generates one micromole of inosine from adenosine per min at 37° C. and expressed as U/L.

### REFERENCES

All documents referenced herein are incorporated by reference, including the following documents for which a full citation is provided below.
Asad, S., et al., (2008) Silicon carbide whisker-mediated embryonic callus transformation of cotton, (gossypium hirsutum L.) and regeneration of salt tolerant plants, Mol. Biotechnol. 40(2): 161-9.
Dmytro, P and Misra S. (2007) Comparison of pathogen-induced expression and efficacy of two amphibian antimicrobial peptides, MsrA2 and temporin A, for engineering wide-spectrum disease resistance in tobacco. Plant Biotech. J. 5, 720-734.
Fischer, R., et al., (2004), Plant-based production of biopharmaceuticals, Current Opinions in Plant Biology 7:152-158.
Flores T. et al., (2002) Ocatin: A novel tuber storage protein from the Andean tuber crop oca with antibacterial and antifungal properties. Plant Physiol April: 128(4): 1291-1302.
Gelvin, S. B., (2003), Agrobacterium-mediated plant transformation: the biology behind the “gene-jockeying” tool. Microbiology and Molecular Biology Reviews, pp 16-37.
Guimaraes et al., 2001. A storage protein from taro shows tuber-specific expression in transgenic potato. Physiol. Plantarum. 111: 182-187.
Jefferson, R. A. (1987). Assaying chimeric genes in plants: The GUS gene fusion system. Plant MOI. Biol. Rep. 5, 387-405.
Karimi, M., De Meyer, B. and Hilson, P. (2005) Modular cloning and expression of tagged fluorescent protein in plant cells. Trends Plant Sci., 10, 103-105.
Koncz, C. and Schell, J. (1986) The promoter of the TL-DNA gene 5 controls the tissue-specific expression of chimeric genes carried by a novel type of Agrobacterium binary vector. Mol. Gen. Genet., 204, 383-396.
Liu, Y. J., Yuan, Y., Zheng, J., Tao, Y. Z., Dong, Z. G., Wang, J. H. and WANG, G. Y. (2004) Signal Peptide of Potato PinII Enhances the Expression of Cry1Ac in Transgenic Tobacco. Acta Biochimica et Biophysica Sinica, 36: 553-558.
Maniatis et al., in Molecular Cloning (A Laboratory Manual), Cold Spring Harbour Laboratory, (1982) p 387 to 389.
Maliga, P., (2004), Plastid transformation in higher plants, Annu. Rev. Plant Biol 55:289-313.
Sanford, J. C., Smith, F. D., Russell, J. A. (1993). Optimizing the biolistic process for different biological applications. Methods Enzymol. 217, 483-509.
Sharma, A. K. et al., (2009), Plants as bioreactors: recent developments and emerging opportunities, Biotechnology Advances 27:811-832.
Shewry, P. R., (2003), Tuber Storage Proteins, Ann Bot 91(7):755-769.
Terpe, K., (2003) Overview of tag protein fusions: from molecular and biochemical fundaments to conventional systems, Appl. Microbiol. Biotechnol. 60:523-533.
Twyman, R. M., et al., (2003) Molecular farming in plants: host systems and expression technology, Trends in Biotechnology Vol 21, No 12, December, pp 570-578.
Ward, W., et al, (2009), Protein purification, Current Analytical Chemistry 5(2):1-21.
Ziolkowski, M. J., (2007) Advancements in biolistics and applications for agriculturally significant crops, MMG445 Basic Biotechnology e Journal 3:34-39.

1. A promoter comprising a nucleotide sequence which is SEQ ID NO:1 or a nucleotide sequence with 80% or greater identity to SEQ ID NO:1 which hybridizes to the complement of SEQ ID NO:1 under stringent conditions.

2. The promoter according to claim 1, wherein the promoter comprises a nucleotide sequence which is:
SEQ ID NO:2;
SEQ ID NO:3;
SEQ ID NO:4; or
a nucleotide sequence with 80% or greater identity to SEQ ID NO:2, SEQ ID NO:3 or SEQ ID NO:4 which hybridizes to the complement of SEQ ID NO:2, SEQ ID NO:3 or SEQ ID NO:4.

3. The promoter according to claim 1 comprising a nucleotide according to SEQ ID NO:1, or comprising a nucleotide sequence with at least 85%, at least 90%, or at least 95% identity to SEQ ID NO. 1.

4. The promoter according to claim 2 comprising a nucleotide according to SEQ ID NO:2, SEQ ID NO:3, or SEQ ID NO:4, or comprising a nucleotide sequence with at least 85%, at least 90%, or at least 95% identity to SEQ ID NO. 2, SEQ ID NO:3 or SEQ ID NO:4.

5. A vector comprising the promoter of claim 1, and optionally a target sequence operatively linked to the promoter.

6. The vector of claim 5, wherein the target sequence encodes an enzyme of the class of orphan diseases for enzyme replacement therapy.

7. The vector of claim 6, wherein the enzyme of a class of orphan disease is adenosine deaminase, preferably human adenosine deaminase; glucocerebrosidase; alpha-galactosidase; alpha-L-iduronidase; alpha-glucosidase; iduronate-2-sulphatase; arylsulphatase B; acid sphingomyelinase; or galactose-6-sulphatase.

8. The vector of claim 5, wherein the target sequence encodes a peptide, preferably an antimicrobial peptide; a cytokine; or a regulatory RNA.

9. A cell transformed with the vector of claim 5.

10. A plant comprising the cell of claim 9.

11. A vegetative propagule of the plant of claim 10.

12. The vegetative propagule of claim 11, wherein the propagule is a tuber, preferably a stem tuber.

13. A method of producing a target protein in a tuber comprising:
transforming a cell with the vector of claim 5;
regenerating a fully functional plant;
expressing the target protein; and
isolating the target protein.

14. A method for producing transformed Oxalis tuberosa comprising:
infecting Oxalis tuberosa nodal stem explant with agrobacterium expressing an expression vector to form an infected nodal explant;
removing callus from infected nodal explant;
inducing a bud on the callus;
inducing shoot formation from the bud; and
producing transformed Oxalis tuberosa from the shoot.

15. The method according to claim 14, wherein the bud is induced on the callus by incubating the callus in BI media, and/or the shoot formation is induced from the callus by growing the bud in BI medium comprising gibberellic acid.

16. A method for producing transformed Oxalis tuberosa comprising:
infecting Oxalis tuberosa stem nodal segments with agrobacterium expressing an expression vector;
inducing a morphogenic callus;
isolating a transformed morphogenetic stem callus;
inducing a shoot bud from the morphogenetic stem callus by growing the bud in a medium comprising gibberellic acid;
germinating the bud;
inducing a shoot from the bud;
germinating and elongating the shoot;
rooting the shoot in rooting media; and
producing a transformed plant.

17. The method according to claim 16, wherein the giberellic acid is GA3 at a concentration of up to about 0.5 mg/L, and/or the rooting media comprises 0.1 mg/L naphthalene acetic acid (NAA).

18. The method according to claim 16, wherein the expression vector comprises a promoter having a nucleotide sequence which is SEQ ID NO:1, SEQ ID NO:2, SEQ ID NO:3, or SEQ ID NO:4.

19. The method according to claim 18, wherein the vector comprises a target sequence operatively linked to the promoter.

20. The method according to claim 19, wherein the target sequence encodes an enzyme of a class of orphan disease.

21. The method according to claim 20, wherein the enzyme of a class of orphan disease is adenosine deaminase, preferably human adenosine deaminase; glucocerebrosidase; alpha-galactosidase; alpha-L-iduronidase; alpha-glucosidase; iduronate-2-sulphatase; arylsulphatase B; acid sphingomyelinase; or galactose-6-sulphatase.

22. The method according to claim 19, wherein the target sequence encodes:
a peptide, preferably an antimicrobial peptide; a cytokine; or a regulatory RNA.

23. A plant produced by the method of claim 16.

24. A vegetative propagule of the plant according to claim 23.


SEQUENCE LISTING (6 sequences)

SEQ ID NO: 1 - DNA, 51 bp, Oxalis tuberosa
ttccgaacacatatagtggacaagttcatcatacataaaagacaacatctg

SEQ ID NO: 2 - DNA, 1307 bp, Oxalis tuberosa
gactcgggttttgtttcttctgactcaaaattctcacaatgttttaattcaaaacactatggctaagaagcaaaagagtctataaattatgaaattgtgtaccgcacatggtaggagaacaaggccgatgacaatttgtctagagtaagagttgaagttgaactcagagttaaatcaactagaatttcatactaatgcttctagagtggtgcgtaaatggtcagaggaagtgttcacataatatgaagacatgatcataaacaaccaactttagacacggttaccaattcattatcggtggtagattcgatataagagatcttagtgatttgagagacagaactttacaaacactcaattggggagcaatctctagttcatgccacttataaaagggtgaaacagattttctactaacaaaaaacattgtaaaatatagccgtgtaggcaaaaatatccgaggtttgtctaaggtgtaatttacagcacatttcctatcttggtctcctgcaacctctacaaattcctaatcaaacttgatagggtattggacagatatcatagaggagcttcctaagtcaaattccctcccctaattctaaacaacgcttttttatgaatcttttcttagacctgtatcatggttgaaattgtcacgttgcgggcgatcattttatttccttcaacttccttgtttagctacaaaaatgtctcaactgttcataaataagaatacaagttgctgatcactcagttgcacttctgatcgcaatagcagagaaatttgtcccgtgcacccaaatcaacggaactgtgcaactagttgccacctcactagcatagaaattttaaggggttggaaatttcaatcctaatctgctaaattgacgggttaaattggccgaacagctagtctctacctttctggacactaacaccccaccaacaacgatgcctttcgttagatctcgttcctaaacgttaaatgccgaggcaggcgaactcttggcaaacccaatgctcacaaatctaatcttctaaggatgtttctcgtcgacacaaatatgaatgactgcaaatgaaatcataagtacttttcttcatcttatgttcttcaggaaacccaacaatctctgtgtcataagttgtgtttttgcctagagtacggaatttgtgcttgattttctctgcttgcttgatccagagaaatcataacaccaatgacataaaccgtaaatgttaaaaatataaatactttccgaacacatatagtggacaagttcatcatacataaaagacaacatctg

SEQ ID NO: 3 - DNA, 1747 bp, Oxalis tuberosa
gattgttcgggaaaaggagtcaaagcacgagaaatgaatgaaatggatataacatagcaaataaatactgctgtaatgctcattgggcagcggtgcgtctgtttggctatattatctaagttgatgattttttaaattcatgaaaagcgaaaaatctaccatatcagatttgagtttagtacaataatgttgatatatcatcgttcaatttcataaacagttggaccggtcgaaaaaggagtcccaaacaagaaatgaatgaaatagaaaaatagatggagatacgccttaaaaggatcagagcacagcctcagatctttgtcaaatactaatagagaacagggtacaattgtcttaacggtcattgggaagcattgaggcgttgcattcctaaaaggcgtgcacacatcatgctccctgccagtgagttttgagcgttggactcgggttttgtttcttctgactcaaaattctcacaatgttttaattcaaaacactatggctaagaagcaaaagagtctataaattatgaaattgtgtaccgcacatggtaggagaacaaggccgatgacaatttgtctagagtaagagttgaagttgaactcagagttaaatcaactagaatttcatactaatgcttctagagtggtgcgtaaatggtcagaggaagtgttcacataatatgaagacatgatcataaacaaccaactttagacacggttaccaattcattatcggtggtagattcgatataagagatcttagtgatttgagagacagaactttacaaacactcaattggggagcaatctctagttcatgccacttataaaagggtgaaacagattttctactaacaaaaaacattgtaaaatatagccgtgtaggcaaaaatatccgaggtttgtctaaggtgtaatttacagcacatttcctatcttggtctcctgcaacctctacaaattcctaatcaaacttgatagggtattggacagatatcatagaggagcttcctaagtcaaattccctcccctaattctaaacaacgcttttttatgaatcttttcttagacctgtatcatggttgaaattgtcacgttgcgggcgatcattttatttccttcaacttccttgtttagctacaaaaatgtctcaactgttcataaataagaatacaagttgctgatcactcagttgcacttctgatcgcaatagcagagaaatttgtcccgtgcacccaaatcaacggaactgtgcaactagttgccacctcactagcatagaaattttaaggggttggaaatttcaatcctaatctgctaaattgacgggttaaattggccgaacagctagtctctacctttctggacactaacaccccaccaacaacgatgcctttcgttagatctcgttcctaaacgttaaatgccgaggcaggcgaactcttggcaaacccaatgctcacaaatctaatcttctaaggatgtttctcgtcgacacaaatatgaatgactgcaaatgaaatcataagtacttttcttcatcttatgttcttcaggaaacccaacaatctctgtgtcataagttgtgtttttgcctagagtacggaatttgtgcttgattttctctgcttgcttgatccagagaaatcataacaccaatgacataaaccgtaaatgttaaaaatataaatactttccgaacacatatagtggacaagttcatcatacataaaagacaacatctg

SEQ ID NO: 4 - DNA, 2238 bp, Oxalis tuberosa
aagcttctcatatctaagctgctgaactagcaagccttgggaaagtcttgtcaaatacgattctaaatgtttcgagtctaacaacagattataccaatgtttgtctacacacctgaatcggacagatgccttcagagggagtttggccaagaattcgactaacattcttgctctgtgtctaggatattgtaacgagccctgtttcatgaaatcaagaaaattagtttttacatatagattgagattgctgaacaaattaagtattacaaaaactttaaaggaaataaagatatagagattggcatcggaaaaaaaaaacctttttgcggcgagggttgaatttgtgaataaggcaaaccaaattagggtttgttgccactatgtgtgtttagatgtgtgcacggatatggctgttcggcgataaatctaagttgattttttaaccttctaaagagcaaaaagtcggctgtttcagactttataaatagttggattgttcgggaaaaggagtcaaagcacgagaaatgaatgaaatggatataacatagcaaataaatactgctgtaatgctcattgggcagcggtgcgtctgtttggctatattatctaagttgatgattttttaaattcatgaaaagcgaaaaatctaccatatcagatttgagtttagtacaataatgttgatatatcatcgttcaatttcataaacagttggaccggtcgaaaaaggagtcccaaacaagaaatgaatgaaatagaaaaatagatggagatacgccttaaaaggatcagagcacagcctcagatctttgtcaaatactaatagagaacagggtacaattgtcttaacggtcattgggaagcattgaggcgttgcattcctaaaaggcgtgcacacatcatgctccctgccagtgagttttgagcgttggactcgggttttgtttcttctgactcaaaattctcacaatgttttaattcaaaacactatggctaagaagcaaaagagtctataaattatgaaattgtgtaccgcacatggtaggagaacaaggccgatgacaatttgtctagagtaagagttgaagttgaactcagagttaaatcaactagaatttcatactaatgcttctagagtggtgcgtaaatggtcagaggaagtgttcacataatatgaagacatgatcataaacaaccaactttagacacggttaccaattcattatcggtggtagattcgatataagagatcttagtgatttgagagacagaactttacaaacactcaattggggagcaatctctagttcatgccacttataaaagggtgaaacagattttctactaacaaaaaacattgtaaaatatagccgtgtaggcaaaaatatccgaggtttgtctaaggtgtaatttacagcacatttcctatcttggtctcctgcaacctctacaaattcctaatcaaacttgatagggtattggacagatatcatagaggagcttcctaagtcaaattccctcccctaattctaaacaacgcttttttatgaatcttttcttagacctgtatcatggttgaaattgtcacgttgcgggcgatcattttatttccttcaacttccttgtttagctacaaaaatgtctcaactgttcataaataagaatacaagttgctgatcactcagttgcacttctgatcgcaatagcagagaaatttgtcccgtgcacccaaatcaacggaactgtgcaactagttgccacctcactagcatagaaattttaaggggttggaaatttcaatcctaatctgctaaattgacgggttaaattggccgaacagctagtctctacctttctggacactaacaccccaccaacaacgatgcctttcgttagatctcgttcctaaacgttaaatgccgaggcaggcgaactcttggcaaacccaatgctcacaaatctaatcttctaaggatgtttctcgtcgacacaaatatgaatgactgcaaatgaaatcataagtacttttcttcatcttatgttcttcaggaaacccaacaatctctgtgtcataagttgtgtttttgcctagagtacggaatttgtgcttgattttctctgcttgcttgatccagagaaatcataacaccaatgacataaaccgtaaatgttaaaaatataaatactttccgaacacatatagtggacaagttcatcatacataaaagacaacatctg

SEQ ID NO: 5 - DNA, 66 bp, Oxalis tuberosa
ttccgaacacatatagtggacaagttcatcatacataaaagacaacatctgaaataacactcgtac

SEQ ID NO: 6 - DNA, 2333 bp, Oxalis tuberosa
aagcttctcatatctaagctgctgaactagcaagccttgggaaagtcttgtcaaatacgattctaaatgtttcgagtctaacaacagattataccaatgtttgtctacacacctgaatcggacagatgccttcagagggagtttggccaagaattcgactaacattcttgctctgtgtctaggatattgtaacgagccctgtttcatgaaatcaagaaaattagtttttacatatagattgagattgctgaacaaattaagtattacaaaaactttaaaggaaataaagatatagagattggcatcggaaaaaaaaaacctttttgcggcgagggttgaatttgtgaataaggcaaaccaaattagggtttgttgccactatgtgtgtttagatgtgtgcacggatatggctgttcggcgataaatctaagttgattttttaaccttctaaagagcaaaaagtcggctgtttcagactttataaatagttggattgttcgggaaaaggagtcaaagcacgagaaatgaatgaaatggatataacatagcaaataaatactgctgtaatgctcattgggcagcggtgcgtctgtttggctatattatctaagttgatgattttttaaattcatgaaaagcgaaaaatctaccatatcagatttgagtttagtacaataatgttgatatatcatcgttcaatttcataaacagttggaccggtcgaaaaaggagtcccaaacaagaaatgaatgaaatagaaaaatagatggagatacgccttaaaaggatcagagcacagcctcagatctttgtcaaatactaatagagaacagggtacaattgtcttaacggtcattgggaagcattgaggcgttgcattcctaaaaggcgtgcacacatcatgctccctgccagtgagttttgagcgttggactcgggttttgtttcttctgactcaaaattctcacaatgttttaattcaaaacactatggctaagaagcaaaagagtctataaattatgaaattgtgtaccgcacatggtaggagaacaaggccgatgacaatttgtctagagtaagagttgaagttgaactcagagttaaatcaactagaatttcatactaatgcttctagagtggtgcgtaaatggtcagaggaagtgttcacataatatgaagacatgatcataaacaaccaactttagacacggttaccaattcattatcggtggtagattcgatataagagatcttagtgatttgagagacagaactttacaaacactcaattggggagcaatctctagttcatgccacttataaaagggtgaaacagattttctactaacaaaaaacattgtaaaatatagccgtgtaggcaaaaatatccgaggtttgtctaaggtgtaatttacagcacatttcctatcttggtctcctgcaacctctacaaattcctaatcaaacttgatagggtattggacagatatcatagaggagcttcctaagtcaaattccctcccctaattctaaacaacgcttttttatgaatcttttcttagacctgtatcatggttgaaattgtcacgttgcgggcgatcattttatttccttcaacttccttgtttagctacaaaaatgtctcaactgttcataaataagaatacaagttgctgatcactcagttgcacttctgatcgcaatagcagagaaatttgtcccgtgcacccaaatcaacggaactgtgcaactagttgccacctcactagcatagaaattttaaggggttggaaatttcaatcctaatctgctaaattgacgggttaaattggccgaacagctagtctctacctttctggacactaacaccccaccaacaacgatgcctttcgttagatctcgttcctaaacgttaaatgccgaggcaggcgaactcttggcaaacccaatgctcacaaatctaatcttctaaggatgtttctcgtcgacacaaatatgaatgactgcaaatgaaatcataagtacttttcttcatcttatgttcttcaggaaacccaacaatctctgtgtcataagttgtgtttttgcctagagtacggaatttgtgcttgattttctctgcttgcttgatccagagaaatcataacaccaatgacataaaccgtaaatgttaaaaatataaatactttccgaacacatatagtggacaagttcatcatacataaaagacaacatctgaaataacactcgtacatgggtgttttcgtattcgaggatgagatcactactactatctccccaactagagtcttcgacagctttgttaacgccga